(19) United States
(12) Patent Application Publication
(10) Pub. No.: US 2023/0187055 A1
(43) Pub. Date: Jun. 15, 2023

(54) SKIN ANALYSIS SYSTEM AND METHOD IMPLEMENTATIONS

(71) Applicant: The Procter & Gamble Company, Cincinnati, OH (US)

(72) Inventors: DISSANAYAKE MUDIYANSELAGE MAHATHMA BANDARA DISSANAYAKE, Mason, OH (US); Paul Jonathan Matts, Addleson (GB); Kaoru MATSUZAKI, Kobe (JP); KUKIZO MIYAMOTO, Kobe (JP)

(21) Appl. No.: 17/890,325

(22) Filed: Aug. 18, 2022

Related U.S. Application Data
(60) Provisional application No. 63/234,245, filed on Aug. 18, 2021.

(51) Int. Cl. G16H 30/40; G16H 20/10

(52) U.S. Cl. CPC G16H 30/40; G16H 20/10

(57) ABSTRACT

Skin analysis systems and methods are described for analyzing user-specific skin data and health data to generate a user-specific skin analysis. User-specific skin data and health data of a user is received by one or more processors, and the health data comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate. A skin analysis learning model analyzes the user-specific skin data and health data to generate a user-specific skin analysis.

### FIELD

The present disclosure generally relates to skin analysis systems and methods, and more particularly to, skin analysis system and method implementations for generating a user-specific skin analysis.

### BACKGROUND

Generally, multiple endogenous factors of human skin, such as sweat and natural oils, have a real-world impact on the overall condition and appearance of a user's skin. Additional exogenous factors, such as wind, humidity, sunlight, and/or usage of various skin-related products, may also affect the condition of a user's skin. Unfortunately, both types of factors contribute to a large number of skin conditions, such as acne, eczema, wrinkles, and general inflammation that can detrimentally impact a user's skin and the user's perception of their skin. However, the user's perception of skin related issues typically does not reflect such underlying endogenous and/or exogenous factors.

Thus a problem arises given the number of endogenous and/or exogenous factors in conjunction with the complexity of skin types, especially when considered across different users, each of whom may be associated with different demographics, races, and ethnicities. This creates a problem in the analysis and treatment of various human skin conditions and characteristics. For example, prior art methods, attempting to aid a user in self-analyzing skin conditions generally lack sufficient information to generate accurate, user-specific analysis, and as a result, offer broad, overly-simplistic recommendations. Further, a user may attempt to empirically experiment with various products or techniques, but without achieving satisfactory results and/or causing possible negative side effects, impacting the condition or otherwise visual appearance of his or her skin.

For the foregoing reasons, there is a need for skin analysis system and method implementations that are configured to accurately generate a user-specific skin analysis.

### SUMMARY

Generally, as described herein, skin analysis system and method implementations are described for generating a user-specific skin analysis. In some aspects, artificial intelligence (AI) based systems and methods herein are configured to train AI models to input user-specific data to generate/predict the user-specific skin analysis. Such AI based systems provide an AI based solution for overcoming problems that arise from the difficulties in identifying and treating various endogenous and/or exogenous factors or attributes affecting the condition of a user's skin.

Generally, the systems as described herein allow a user to submit user-specific data to a server(s) (e.g., including its one or more processors), or otherwise a computing device (e.g., such as locally on the user's mobile device), where the server(s) or user computing device, implements or executes one or more skin analysis learning model(s) configured to generate a user-specific skin analysis. In certain aspects, the one or more skin analysis learning model(s) are trained with training data of potentially thousands of instances (or more) of user-specific data regarding skin regions of respective individuals. The skin analysis learning model(s) receives the user-specific data as input, and generates a user-specific analysis that is designed to address (e.g., identify and/or treat) at least one feature of the user's skin region. For example, the user-specific data may comprise responses or other inputs indicative of sugar intake and/or other skin factors of a specific user's skin regions, and one or more image(s)/video(s) of the user's skin regions. Moreover, in certain aspects, the skin analysis learning model(s) generates a user-specific skin analysis comprising one or more selected from the group comprising: (1) a skin condition, (2) a holistic score to be defined based on at least skin and health data, (3) a skin product recommendation, (4) a skin product usage recommendation, (5) a supplemental product recommendation, (6) a supplemental product usage recommendation, (7) a habit recommendation, and (8) a skin forecast corresponding to the user's skin region based on the skin data and the health data of the user

Each of the user submitted images and/or videos may be received at a server(s) (e.g., including its one or more processors) (also referenced herein as an “imaging server”), or otherwise a computing device (e.g., such as locally on the user's mobile device), where the imaging server(s) or user computing device, implements or executes the skin analysis learning model(s). In certain aspects, the skin analysis learning model may be an AI based model trained with pixel data of potentially 10,000s (or more) images depicting skin regions of respective individuals. The AI based skin analysis learning model may generate a user-specific analysis designed to address (e.g., identify and/or treat) at least one feature identifiable within the pixel data comprising the user's skin region. For example, a portion of a user's skin region can comprise pixels or pixel data indicative of eczema, acne, wrinkles, inflammation, and/or other skin factors of a specific user's skin regions. In some aspects, the user-specific skin analysis and recommendation(s)/score(s)/forecast(s) may be transmitted via a computer network to a user computing device of the user for rendering on a display screen. In other aspects, no transmission to the imaging server of the image of the user occurs, where the user-specific skin analysis and recommendation(s)/score(s)/forecast(s) may instead be generated by the AI based skin analysis learning model(s), executing and/or implemented locally on the user's mobile device and rendered, by a processor of the mobile device, on a display screen of the mobile device. In various aspects, such rendering may include graphical representations, overlays, annotations, and the like for addressing the feature in the pixel data.

More specifically, as described herein, a user-specific skin analysis method is disclosed for generating a user-specific skin analysis. The user-specific skin analysis method comprises: receiving, by one or more processors, a skin data of the user; receiving, by the one or more processors, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and analyzing, by one or more skin analysis learning models, the skin data of the user and the health data of the user to generate, a user-specific skin analysis.

In addition, as described herein, a user-specific skin analysis system is disclosed. The user-specific skin analysis system is configured to generate a user-specific skin analysis. The user-specific skin analysis system comprises: one or more processors; and an analysis application (app) comprising computing instructions configured to execute on the one or more processors; and one or more skin analysis learning models, accessible by the analysis app, wherein the computing instructions of the analysis app when executed by the one or more processors, cause the one or more processors to: receive a skin data of the user, receive a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate, and analyze, by the one or more skin analysis learning models, the skin data of the user and the health data of the user to generate, a user-specific skin analysis.

Further, as described herein, a tangible, non-transitory computer-readable medium storing instructions for generating a user-specific skin analysis is disclosed. The instructions, when executed by one or more processors, may cause the one or more processors to: receive, at an analysis application (app) executing on one or more processors, a skin data of the user; receive, at the analysis app, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and analyze, by the one or more skin analysis learning models accessible by the analysis app, the skin data of the user and the health data of the user to generate, a user-specific skin analysis.

In accordance with the above, and with the disclosure herein, the present disclosure includes improvements in computer functionality or in improvements to other technologies at least because the disclosure describes that, e.g., a server, or otherwise computing device (e.g., a user computer device), is improved where the intelligence or deterministic capabilities of the server or computing device are enhanced by the skin analysis learning model(s). The skin analysis learning model(s), executing on the server or computing device, is able to more accurately identify, based on user-specific skin data and health data, a user-specific skin analysis than conventional techniques. In fact, the skin analysis learning model(s) of the present disclosure may receive from a user one or more input images of the user's skin and one or more answers to a questionnaire, and using image/video processing techniques or questionnaire or other devices, may determine the user-specific skin data and health data of the user. As a result, the skin analysis learning model(s) may quickly and efficiently provide the user-specific skin analysis in a manner that was previously unachievable by conventional techniques.

Moreover, the present disclosure includes improvements in computer functionality or in improvements to other technologies at least because the disclosure describes that, e.g., a server, or otherwise computing device (e.g., a user computer device), is improved where the intelligence or predictive ability of the server or computing device is enhanced by trained (e.g., machine learning trained) skin analysis learning model(s). The skin analysis learning model(s), executing on the server or computing device, is able to more accurately identify, based on skin data and health data of other individuals, a user-specific skin analysis designed to address at least one feature of the user's skin or skin region. That is, the present disclosure describes improvements in the functioning of the computer itself or “any other technology or technical field” because a server or user computing device is enhanced with a plurality of training data (e.g., potentially thousands of instances (or more) of skin data and health data regarding skin regions of respective individuals) to accurately predict, detect, or determine user-specific skin analysis based on skin data and health data of a user, such as or derived from newly provided customer responses/inputs/images. This improves over the prior art at least because existing systems lack such predictive or classification functionality and are simply not capable of accurately analyzing skin data and health data of a user to output a predictive result to address at least one feature of the user's skin or skin region.

Specifically, the systems and methods of the present disclosure feature improvements over conventional techniques by the use of specific health data together with skin data. By the use of the specific health data together with skin data, the present invention provides improved accuracy of skin analysis, compared to prior analysis, for example, that using skin data only. Such specific health data comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate. In certain aspects, the health data is selected from the group consisting of: (1) the body water content or amount, (2) the intracellular-to-extracellular water ratio, (3) the body mass index (BMI), and/or mixtures thereof, or in some aspects, the health data is selected from the group consisting of: (1) the body water content or amount, (2) the intracellular-to-extracellular water ratio, and/or mixtures thereof, in view of providing further improved accuracy of skin analysis. It was surprisingly found that these specific health data provides improved accuracy of skin analysis, compared to other health data such as percent body fat, when used together with skin data. Additionally, the systems and methods of the present disclosure feature improvements over conventional techniques by training the skin analysis learning model(s) with a plurality of training data related to skin data and health data of a plurality of individuals. The training data generally includes an individual's self-assessment of the individual's skin in the form of textual questionnaire responses for each of the plurality of individuals, and images of the user's skin region(s) captured with an imaging device. Once trained using the training data, the skin analysis learning model(s) provide high-accuracy skin analysis predictions for a user to a degree that is unattainable using conventional techniques. In fact, using the health data together with the skin data, the present disclosure provides improved accuracy of the resulting user-specific skin analysis compared to prior art analysis techniques that, for example, only utilize skin data.

For similar reasons, the present disclosure relates to improvement to other technologies or technical fields at least because the present disclosure describes or introduces improvements to computing devices in the skin care field and skin care products field, whereby the trained skin analysis learning model(s) executing on the computing devices and/or imaging device(s) improve the underlying computer device (e.g., server(s) and/or user computing device), where such computer devices are made more efficient by the configuration, adjustment, or adaptation of a given machine-learning network architecture. For example, in some aspects, fewer machine resources (e.g., processing cycles or memory storage) are used by decreasing computational resources by decreasing machine-learning network architecture needed to analyze images, including by reducing depth, width, image size, or other machine-learning based dimensionality requirements. Such reduction frees up the computational resources of an underlying computing system, thereby making it more efficient.

In addition, the present disclosure includes applying certain of the claim elements with, or by use of, a particular machine, e.g., an imaging device, which generates training data that may be used to train the skin analysis learning model(s).

In addition, the present disclosure includes specific features other than what is well-understood, routine, conventional activity in the field, or adding unconventional steps that confine the claim to a particular useful application, e.g., analyzing user-specific skin data and health data to generate a user-specific skin analysis designed to address (e.g., identify and/or treat) at least one feature of the user's skin.

Advantages will become more apparent to those of ordinary skill in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an aspect of a particular aspect of the disclosed system and methods, and that each of the Figures is intended to accord with a possible aspect thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present aspects are not limited to the precise arrangements and instrumentalities shown, wherein:

FIG. 1 illustrates an example user-specific skin analysis system configured to analyze user-specific skin data and health data to generate a user-specific skin analysis, in accordance with various aspects disclosed herein.

FIG. 2 is an example flow diagram depicting the operation of the skin analysis learning model from the example user-specific skin analysis system of FIG. 1, in accordance with various aspects disclosed herein.

FIG. 3 illustrates an embodiment of the skin analysis learning model from the example user-specific skin analysis system of FIG. 1, in accordance with various aspects disclosed herein.

FIG. 4 illustrates an example user-specific skin analysis method for generating a user-specific skin analysis, in accordance with various aspects disclosed herein.

FIG. 5 illustrates an example user interface as rendered on a display screen of a user computing device in accordance with various aspects disclosed herein.

The Figures depict preferred aspects for purposes of illustration only. Alternative aspects of the systems and methods illustrated herein may be employed without departing from the principles of the invention described herein.

### DETAILED DESCRIPTION OF THE INVENTION

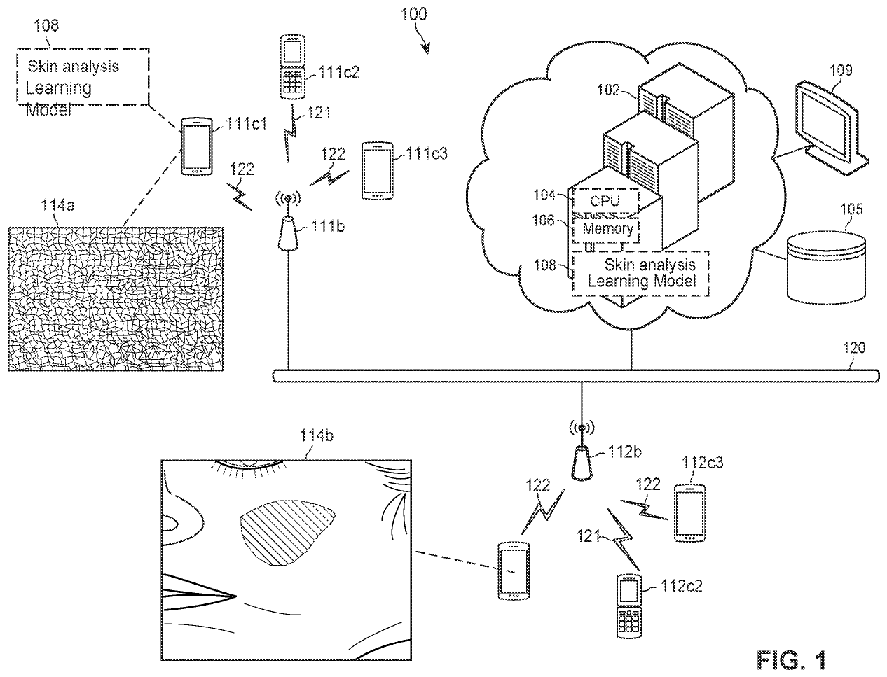

FIG. 1 illustrates an example user-specific skin analysis system 100 configured to analyze user-specific skin data and health data to generate a user-specific skin analysis, in accordance with various aspects disclosed herein. Generally, as referred to herein, the user-specific skin data and health data may include and/or be derived from user responses/inputs related to questions/prompts presented to the user via a display and/or user interface of a user computing device that are directed to the condition of the user's skin and/or images captured by the user computing device that depict a skin region of the user's skin. For example, the user-specific health data may include a user response indicating a sugar intake level of the user over a particular period of time (e.g., daily, weekly, etc.). As another, the user-specific skin data and health data may include data obtained by processing one or more images of a skin region of the user, as captured by the user with a user computing device. Of course, as described herein, the images of the user's skin region may include still images (e.g., individual image frames) and/or video (e.g., multiple image frames) captured using an imaging device (e.g., a user computing/mobile device).

In the example aspect of FIG. 1, the AI based system 100 includes server(s) 102, which may comprise one or more computer servers. In various aspects server(s) 102 comprise multiple servers, which may comprise multiple, redundant, or replicated servers as part of a server farm. In still further aspects, server(s) 102 may be implemented as cloud-based servers, such as a cloud-based computing platform. For example, server(s) 102 may be any one or more cloud-based platform(s) such as MICROSOFT AZURE, AMAZON AWS, or the like. Server(s) 102 may include one or more processor(s) 104, one or more computer memories 106, and a skin analysis learning model 108.

The memories 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memorie(s) 106 may store an operating system (OS) (e.g., Microsoft Windows, Linux, UNIX, etc.) capable of facilitating the functionalities, apps, methods, or other software as discussed herein. The memorie(s) 106 may also store the skin analysis learning model 108, which may be a machine learning model, trained on various training data (e.g., potentially thousands of instances (or more) of user-specific data regarding skin regions of respective individuals) and, in certain aspects, images (e.g., images 114a, 114b), as described herein. Additionally, or alternatively, the skin analysis learning model 108 may also be stored in database 105, which is accessible or otherwise communicatively coupled to server(s) 102. In addition, memories 106 may also store machine readable instructions, including any of one or more application(s) (e.g., a scalp and hair application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, an AI based machine learning model or component, such as the skin analysis learning model 108, where each may be configured to facilitate their various functionalities discussed herein. It should be appreciated that one or more other applications may be envisioned and that are executed by the processor(s) 104.

The processor(s) 104 may be connected to the memories 106 via a computer bus responsible for transmitting electronic data, data packets, or otherwise electronic signals to and from the processor(s) 104 and memories 106 in order to implement or perform the machine readable instructions, methods, processes, elements or limitations, as illustrated, depicted, or described for the various flowcharts, illustrations, diagrams, figures, and/or other disclosure herein.

Processor(s) 104 may interface with memory 106 via the computer bus to execute an operating system (OS). Processor(s) 104 may also interface with the memory 106 via the computer bus to create, read, update, delete, or otherwise access or interact with the data stored in memories 106 and/or the database 104 (e.g., a relational database, such as Oracle, DB2, MySQL, or a NoSQL based database, such as MongoDB). The data stored in memories 106 and/or database 105 may include all or part of any of the data or information described herein, including, for example, training data (e.g., as collected by user computing devices 111c1-111c3 and/or 112c1-112c3); images and/or user images (e.g., including images 114a, 114b); and/or other information and/or images of the user, including demographic, age, race, skin type, or the like, or as otherwise described herein.

The server(s) 102 may further include a communication component configured to communicate (e.g., send and receive) data via one or more external/network port(s) to one or more networks or local terminals, such as computer network 120 and/or terminal 109 (for rendering or visualizing) described herein. In some aspects, the server(s) 102 may include a client-server platform technology such as ASP.NET, Java J2EE, Ruby on Rails, Nodejs, a web service or online API, responsive for receiving and responding to electronic requests. The server(s) 102 may implement the client-server platform technology that may interact, via the computer bus, with the memories(s) 106 (including the applications(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various aspects, the server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some aspects, computer network 120 may comprise a private network or local area network (LAN). Additionally, or alternatively, computer network 120 may comprise a public network such as the Internet.

The server(s) 102 may further include or implement an operator interface configured to present information to an administrator or operator and/or receive inputs from the administrator or operator. As shown in FIG. 1, an operator interface may provide a display screen (e.g., via terminal 109). The server(s) 102 may also provide I/O components (e.g., ports, capacitive or resistive touch sensitive input panels, keys, buttons, lights, LEDs), which may be directly accessible via, or attached to, imaging server(s) 102 or may be indirectly accessible via or attached to terminal 109. According to some aspects, an administrator or operator may access the server 102 via terminal 109 to review information, make changes, input training data or images, initiate training of the skin analysis learning model 108, and/or perform other functions.

As described herein, in some aspects, the server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 1, the server(s) 102 are communicatively connected, via computer network 120 to the one or more user computing devices 111c1-111c3 and/or 112c1-112c3 via base stations 111b and 112b. In some aspects, base stations 111b and 112b may comprise cellular base stations, such as cell towers, communicating to the one or more user computing devices 111c1-111c3 and 112c1-112c3 via wireless communications 121 based on any one or more of various mobile phone standards, including NMT, GSM, CDMA, UMMTS, LTE, 5G, or the like. Additionally, or alternatively, base stations 111b and 112b may comprise routers, wireless switches, or other such wireless connection points communicating to the one or more user computing devices 111c1-111c3 and 112c1-112c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 and/or 112c1-112c3 may comprise mobile devices and/or client devices for accessing and/or communications with the server(s) 102. Such client devices may comprise one or more mobile processor(s) and/or an imaging device for capturing images, such as images as described herein (e.g., images 114a, 114b). In various aspects, user computing devices 111c1-111c3 and/or 112c1-112c3 may comprise a mobile phone (e.g., a cellular phone), a tablet device, a personal data assistance (PDA), or the like, including, by non-limiting example, an APPLE iPhone or iPad device or a GOOGLE ANDROID based mobile phone or table.

In additional aspects, user computing devices 111c1-111c3 and/or 112c1-112c3 may comprise a retail computing device. A retail computing device may comprise a user computer device configured in a same or similar manner as a mobile device, e.g., as described herein for user computing devices 111c1-111c3 and 112c1-112c3, including having a processor and memory, for implementing, or communicating with (e.g., via server(s) 102), a skin analysis learning model 108 as described herein. Additionally, or alternatively, a retail computing device may be located, installed, or otherwise positioned within a retail environment to allow users and/or customers of the retail environment to utilize the AI based systems and methods on site within the retail environment. For example, the retail computing device may be installed within a kiosk for access by a user. The user may then provide responses to a questionnaire and/or upload or transfer images (e.g., from a user mobile device) to the kiosk to implement the AI based systems and methods described herein. Additionally, or alternatively, the kiosk may be configured with a camera to allow the user to take new images (e.g., in a private manner where warranted) of himself or herself for upload and transfer. In such aspects, the user or consumer himself or herself would be able to use the retail computing device to receive and/or have rendered a user-specific skin analysis related to the user's skin region, as described herein, on a display screen of the retail computing device.

Additionally, or alternatively, the retail computing device may be a mobile device (as described herein) as carried by an employee or other personnel of the retail environment for interacting with users or consumers on site. In such aspects, a user or consumer may be able to interact with an employee or otherwise personnel of the retail environment, via the retail computing device (e.g., by providing responses to a questionnaire, transferring images from a mobile device of the user to the retail computing device, or by capturing new images by a camera of the retail computing device), to receive and/or have rendered a user-specific skin analysis related to the user's skin region, as described herein, on a display screen of the retail computing device.

In various aspects, the one or more user computing devices 111c1-111c3 and/or 112c1-112c3 may implement or execute an operating system (OS) or mobile platform such as APPLE's iOS and/or GOOGLE's ANDROID operation system. Any of the one or more user computing devices 111c1-111c3 and/or 112c1-112c3 may comprise one or more processors and/or one or more memories for storing, implementing, or executing computing instructions or code, e.g., a mobile application or a home or personal assistant application, as described in various aspects herein. As shown in FIG. 1, the skin analysis learning model 108 and/or an analysis application as described herein, or at least portions thereof, may also be stored locally on a memory of a user computing device (e.g., user computing device 111c1).

User computing devices 111c1-111c3 and/or 112c1-112c3 may comprise a wireless transceiver to receive and transmit wireless communications 121 and/or 122 to and from base stations 111b and/or 112b. In various aspects, user-specific skin data and health data (e.g., user responses/inputs to questionnaire(s) presented on user computing device 111c1, and pixel based images captured by user computing device 111c1 (e.g., images 114a, 114b)) may be transmitted via computer network 120 to the server(s) 102 for training of model(s) (e.g., skin analysis learning model 108) and/or analysis as described herein.

In addition, the one or more user computing devices 111c1-111c3 and/or 112c1-112c3 may include an imaging device and/or digital video camera for capturing or taking digital images and/or frames (e.g., images 114a, 114b). Each digital image may comprise pixel data for training or implementing model(s), such as AI or machine learning models, as described herein. For example, an imaging device and/or digital video camera of, e.g., any of user computing devices 111c1-111c3 and/or 112c1-112c3, may be configured to take, capture, or otherwise generate digital images (e.g., pixel based images 114a, 114b) and, at least in some aspects, may store such images in a memory of a respective user computing devices. Additionally, or alternatively, such digital images may also be transmitted to and/or stored on memorie(s) 106 and/or database 105 of server(s) 102.

Still further, each of the one or more user computer devices 111c1-111c3 and/or 112c1-112c3 may include a display screen for displaying graphics, images, text, products, user-specific treatments, data, pixels, features, and/or other such visualizations or information as described herein. In various aspects, graphics, images, text, products, user-specific treatments, data, pixels, features, and/or other such visualizations or information may be received from the server(s) 102 for display on the display screen of any one or more of user computer devices 111c1-111c3 and/or 112c1-112c3. Additionally, or alternatively, a user computing device may comprise, implement, have access to, render, or otherwise expose, at least in part, an interface or a guided user interface (GUI) for displaying text and/or images on its display screen.

In some aspects, computing instructions and/or applications executing at the server (e.g., server(s) 102) and/or at a mobile device (e.g., mobile device 111c1) may be communicatively connected for analyzing user-specific skin data and health data comprising one or more of (1) a body water content or amount, (2) an intracellular-to-extracellular water ratio, (3) a body mass index (BMI), (4) a blood marker, (5) a sugar intake level, (6) a heart rate variability, or (7) a heart rate to generate a user-specific skin analysis, as described herein. For example, one or more processors (e.g., processor(s) 104) of server(s) 102 may be communicatively coupled to a mobile device via a computer network (e.g., computer network 120). For ease of discussion, the skin data of the user and the health data of the user may be collectively referenced herein as “user-specific data”.

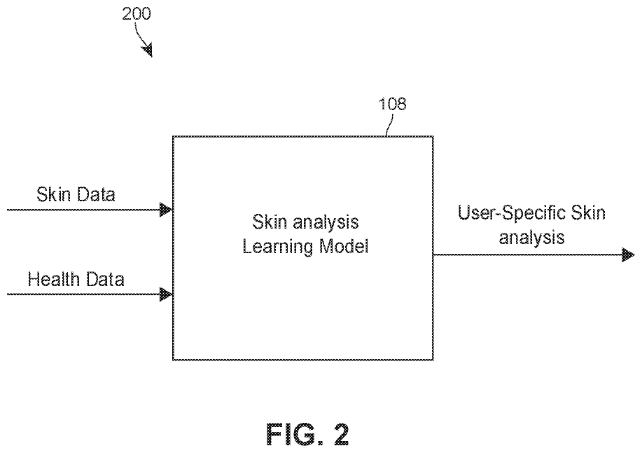

FIG. 2 is an example flow diagram 200 depicting the operation of the skin analysis learning model 108 from the example user-specific skin analysis system 100 of FIG. 1, in accordance with various aspects disclosed herein. Generally, the skin analysis learning model 108 receives skin data and health data of a user (the “user-specific data”) as input and outputs a user-specific skin analysis. In certain aspects, the skin analysis learning model 108 may be or include one or more rules-based models, while in some aspects, the model 108 may be and/or otherwise include one or more AI based models. As such, the example flow diagram 200 may also depict an example training sequence of the skin analysis learning model 108, wherein the model 108 receives training data comprising skin data and health data of multiple respective users as input, and outputs user-specific skin analysis corresponding to each of the respective users as output.

In aspects where the user-specific data is utilized to train the skin analysis learning model 108, at least a portion of the user-specific data may be in the form of user submitted responses to a questionnaire. Generally, the questionnaire responses may train the skin analysis learning model 108 by enabling the model 108 to determine various correlations between the responses and the user-specific skin analysis. Further, in certain aspects, the skin analysis learning model 108 may be configured to generate one or more outputs in addition to and/or as part of the user-specific skin analysis in response to receiving the responses from the user, such as (1) a skin condition, (2) a holistic score to be defined based on at least skin and health data, (3) a skin product recommendation, (4) a skin product usage recommendation, (5) a supplemental product recommendation, (6) a supplemental product usage recommendation, (7) a habit recommendation, and/or (8) a skin forecast corresponding to the user's skin region.

In addition, the user-specific data submitted by a user as inputs to the skin analysis learning model 108 may include image data of the user. Specifically, in certain aspects, the image data may include a digital image depicting at least a portion of a skin region of the user. Each image may be used to train and/or execute the skin analysis learning model 108 for use across a variety of different users having a variety of different skin region features. For example, as illustrated for images 114a, 114b in FIG. 1, the skin regions of the users of these images comprise skin region features of the respective users' skin that are identifiable with the pixel data of the images 114a, 114b. These skin region features include, for example, skin inflammation and skin redness, which the skin analysis learning model 108 may identify within the images 114a, 114b, and may use to generate a user-specific skin analysis for the users represented in the images 114a, 114b, as described herein.

A user may execute an analysis application (app), which in turn, may display a user interface that may include sections/prompts for a user to input portions of the user-specific data. When the user provides responses to the questionnaire prompts, the skin analysis learning model 108 may analyze the user-specific data to generate the user-specific skin analysis, and the analysis app may render a user interface that may include the user-specific skin analysis, indications of the user responses, and/or the user-specific data.

As previously mentioned, the skin analysis learning model 108 executing on the analysis app may be an AI based model. Accordingly, the skin analysis learning model 108 may be trained using a supervised machine learning program or algorithm, such as a multivariate regression analysis or neural network. Generally, machine learning may involve identifying and recognizing patterns in existing data (such as generating skin analysis corresponding to one or more features of the skin regions of respective individuals) in order to facilitate making predictions or identification for subsequent data (such as using the model on new user-specific data in order to determine or generate a skin analysis corresponding to the skin region of a user). Machine learning model(s), such as the skin analysis learning model 108 described herein for some aspects, may be created and trained based upon example data (e.g., “training data” and related user-specific data) inputs or data (which may be termed “features” and “labels”) in order to make valid and reliable predictions for new inputs, such as testing level or production level data or inputs.

In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In certain aspects, the skin analysis learning model 108 may be trained using multiple supervised machine learning techniques, and may additionally or alternatively be trained using one or more unsupervised machine learning techniques. In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

For example, in certain aspects, the skin analysis learning model may employ a neural network, which may be a convolutional neural network, a deep learning neural network, or a combined learning module or program that learns in two or more features or feature datasets (e.g., user-specific data) in particular areas of interest. The machine learning programs or algorithms may also include natural language processing, semantic analysis, automatic reasoning, support vector machine (SVM) analysis, decision tree analysis, random forest analysis, K-Nearest neighbor analysis, naïve Bayes analysis, clustering, reinforcement learning, and/or other machine learning algorithms and/or techniques. In some aspects, the artificial intelligence and/or machine learning based algorithms may be included as a library or package executed on the server(s) 102. For example, libraries may include the TENSORFLOW based library, the PYTORCH library, and/or the SCIKIT-LEARN Python library.

Regardless, training the skin analysis learning model may also comprise retraining, relearning, or otherwise updating models with new, or different, information, which may include information received, ingested, generated, or otherwise used over time. Moreover, in various aspects, the skin analysis learning model 108 may be trained, by one or more processors (e.g., one or more processor(s) 104 of server(s) 102 and/or processors of a computer user device, such as a mobile device) with the pixel data of a plurality of training images (e.g., images 114a, 114b) of the skin regions of respective individuals. In these aspects, the skin analysis learning model 108 may additionally be configured to generate a user-specific analysis corresponding to one or more features of the skin regions of each respective individual in each of the plurality of training images.

Generally, the skin data included as part of the user-specific data may be derived from user-submitted images of the user's skin (e.g., a skin region). A user may submit one or more images of the user's skin, and the skin analysis learning model 108 may apply various image processing techniques to the submitted images to determine any number of skin characteristics. For example, a user may capture/submit an image of a skin region that includes redness and inflammation (e.g., image 114b). The skin analysis learning model 108 may receive the image and apply image processing techniques, such as but not limited to, image classification, object detection, object tracking, semantic segmentation, instance segmentation, edge detection, anisotropic diffusion, pixilation, point feature mapping, and/or other suitable image processing techniques or combinations thereof. As a result, the skin analysis learning model 108 may generate features or characteristics of the user's submitted image that enable the model 108 to determine a user-specific skin analysis based on correlations between/among the features/characteristics and the known skin analysis.

Additionally, or alternatively, the skin data included as part of the user-specific data may be submitted by the user as part of the responses to the questionnaire. For example, the analysis app may query the user whether or not the user experiences any skin issues, such as dryness, itchiness, redness, etc. In response to the user selecting one or more of the skin issues, the analysis app may further request that the user select one or more options (e.g., via rendering the options as part of the analysis app display) indicating a degree of severity related to the user's indicated skin issue. A user may indicate each applicable skin issue and/or corresponding degree of severity through, for example, interaction with a user interface of the analysis app executing on the user's computing device (e.g., user computing device 111c1). For each skin issue indicated by the user, the skin analysis learning model 108 may incorporate the indicated skin issue and/or corresponding degree of severity as part of the analysis of the user-specific data to generate the user-specific skin analysis.

Moreover, the health data included as part of the user-specific data may also be derived from user-submitted images of the user's skin (e.g., a skin region) and/or may be submitted by the user as part of the responses to the questionnaire. In certain aspects, the user may submit images of the user's skin as input into the skin analysis learning model 108. In these aspects, the skin analysis learning model 108 may apply various image processing techniques, as previously mentioned, to determine any number of health characteristics of the user. For example, the user may submit an image that features a healthy portion of the user's skin, and as a result, the skin analysis learning model 108 may generate health characteristics based on the image that allow the skin analysis learning model 108 to output the user-specific skin analysis. Specifically, the skin analysis learning model 108 may generate health characteristics that include, but are not limited to, a user's body water content/amount, body mass index (BMI), intercellular-to-extracellular water ratio, sugar levels, heart rate, heart rate variability, and/or other suitable health characteristics or combinations thereof. In any event, the user may also submit the health data as part of the questionnaire displayed by the analysis app, as previously described.

Of course, it is to be understood that the questions/prompts presented as part of the questionnaire displayed to a user in the analysis app may include any suitable input option for a user to input responses, such as a sliding scale and/or a manually-entered (e.g., by typing on a keyboard or virtually rendered keyboard on the user's mobile device) numerical value or character string indicating a skin issue or otherwise response.

The user-specific analysis output by the skin analysis learning model 108 may generally include a textual and/or graphical output corresponding to the skin condition determined by the model 108. For example, the user-specific analysis may include a graphical rendering that includes textual characters conveying to a user viewing the display that the skin region included in the submitted image and/or indicated by the user's responses to the questionnaire may have skin redness as a result of inflammation (e.g., image 114b). The user-specific analysis may also include any number of various indications, such as a skin score, which may generally indicate the current quality/condition of the user's skin region featured in the image(s) and/or indicated in the user's responses to the questionnaire. The skin score may indicate that the current quality/condition of the user's skin region is, for example, a 3.5 out of a potential maximum score of 4, which may represent that the user's skin region is relatively healthy. However, it is to be understood that the skin score (and any other score or indication included as part of the user-specific skin analysis) may be represented to a user as a graphical rendering, an alphanumerical value, a color value, and/or any other suitable representation or combinations thereof.

Moreover, the AI based learning model may also generate, a skin score description that may inform a user about their received skin score (e.g., represented by the graphical score). The analysis app may render the scalp score description as part of the user interface when the skin analysis learning model completes the analysis of the user-specific data. The scalp score description may include a description of, for example, a predominant skin issue/condition leading to a reduced score, endogenous/exogenous factors causing skin issues, and/or any other information or combinations thereof. As an example, the skin score description may inform a user that their skin region is slightly inflamed, and as a result, the user may experience undesired skin warmth and discomfort. Further in this example, the skin score description may convey to the user that irritants such as dry air may cause and/or otherwise contribute to the inflammation. It is to be appreciated that the above description related to the skin score may generally apply to any output of the skin analysis learning model 108 included as part of the user-specific skin analysis, such as (1) a skin condition, (2) a holistic score to be defined based on at least skin and health data, (3) a skin product recommendation, (4) a skin product usage recommendation, (5) a supplemental product recommendation, (6) a supplemental product usage recommendation, (7) a habit recommendation, and/or (8) a skin forecast corresponding to the user's skin region.

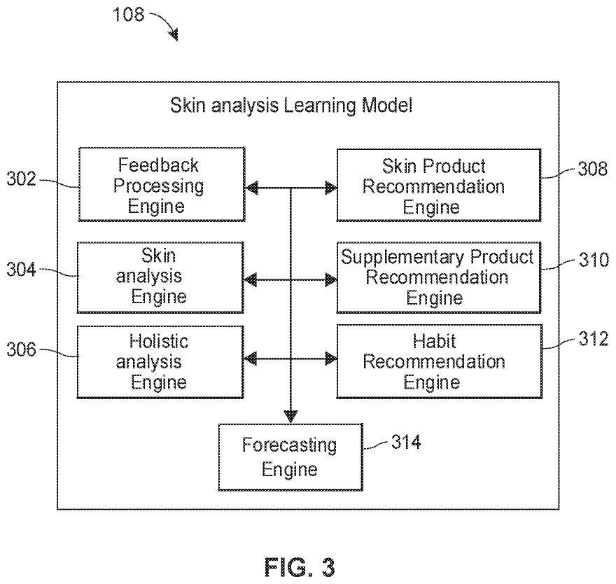

FIG. 3 illustrates an embodiment of the skin analysis learning model 108 from the example user-specific skin analysis system 100 of FIG. 1, in accordance with various aspects disclosed herein. In the embodiment illustrated in FIG. 3, the skin analysis learning model 108 includes seven individual machine learning models (referenced herein as “engines”) that may be configured to operate together. More specifically, the embodiment of the skin analysis learning model 108 may comprise an ensemble model comprising multiple AI models or sub-models that are configured to operate together.

Additionally, or alternatively, the skin analysis learning model 108 may comprise a transfer learning based set of AI models, where transfer learning comprises transferring knowledge from one model to another (e.g., outputs of one model are used as inputs to another model). Using transfer learning, a particular task (e.g., identification, classification, and/or or prediction) may be solved using full or part of an already pre-trained model on a different task. FIG. 3 illustrates such an ensemble AI model and/or transfer learning based AI model, which may comprise the skin analysis learning model 108. It is to be understood, however, that other AI models (not requiring ensemble based learning or transfer learning based learning) may be used.

In particular, the skin analysis learning model 108 illustrated in FIG. 3 includes a feedback processing engine 302, a skin analysis engine 304, a holistic analysis engine 306, a skin product recommendation engine 308, a supplementary product recommendation engine 310, a habit recommendation engine 312, and a forecasting engine 314. Each of these individual engines 302-314 may operate sequentially, and the output of one engine may serve as the input to another subsequent engine. For example, the feedback processing engine 302 may be trained with the health data of the respective individuals to output respective health values, and the feedback processing engine 302 may be configured to receive the health data of the user and to output a user health value based on the health data of the user. The user health value may be a numerical or otherwise value representing a general health assessment of the user based on the health data provided by the user (e.g., via responses and/or images).

In turn, the skin analysis engine 304 may be configured to receive the user health value of the user from the feedback processing engine 302 and to output a skin condition value (e.g., a skin score) of the user based on the user health value. As previously mentioned, the skin condition value or skin score of the user may generally correspond to a numerical or otherwise value representing the current quality/condition of the user's skin. The skin analysis engine 304 may be trained with the respective health values received from the feedback processing engine 302 to output respective skin condition values.

The holistic analysis engine 306 may be configured to receive the skin condition value of the user from the skin analysis engine 304 and to output a holistic score of the user based on the skin condition value of the user. The holistic score of the user may generally correspond to a single (“holistic”) numerical or otherwise score that represents the overall health score of the user's skin based on the user's skin data and health data (e.g., sugar intake, heart rate, etc.). The holistic analysis engine 306 may be trained with the respective skin condition values received from the skin analysis engine 304 to output respective holistic scores.

Thereafter, the skin product recommendation engine 308 may receive the holistic score of the user from the holistic analysis engine 306. The skin product recommendation engine 308 may be configured to receive the skin condition value of the user from the skin analysis engine 304 and the holistic score of the user from the holistic analysis engine 306 and to output a skin product recommendation of the user based on the skin condition value of the user and the holistic score of the user. In certain aspects, the skin product recommendation comprises a skin product usage recommendation configured to provide the user with product application instructions corresponding to a skin product identified as part of the skin product recommendation. For example, the skin product recommendation engine 308 may output a recommendation suggesting that a user apply moisturizing lotion to the user's skin region in order to alleviate dryness/itchiness. As another example, the skin product recommendation engine 308 may output a recommendation suggesting that a user apply anti-wrinkle products to the user's skin region in order to minimize/reduce wrinkles identified on the user's skin region. The skin product recommendation engine 308 may be trained with the respective skin condition values from the skin analysis engine 304 and the respective holistic scores received from the holistic analysis engine 306 to output respective skin product recommendations.

The supplementary product recommendation engine 310 may receive the holistic score from the holistic analysis engine 306 in order to output a supplementary product recommendation based on the holistic score of the user. The supplementary product recommendation may generally be and/or include a supplementary product relative to the skin product recommended by the skin product recommendation engine 308, such as vitamins or other products that may supplement a skin care regimen. Accordingly, in certain aspects, the supplementary product recommendation comprises a supplementary product usage recommendation configured to provide the user with product application instructions corresponding to a supplementary product identified as part of the supplementary product recommendation. For example, the supplementary product recommendation engine 310 may output a recommendation suggesting that the user take a vitamin D supplement in order to generally improve their skin health. The supplementary product recommendation engine 310 may be trained with the respective holistic scores received from the holistic analysis engine 306 to output respective supplementary product recommendations.

The habit recommendation engine 312 may receive the holistic score of the user from the holistic analysis engine 306, and may output a habit recommendation of the user based on the holistic score of the user. The habit recommendation may generally be and/or include a recommended habit and/or corresponding product intended to improve the overall health (and as a result, the holistic score) of the user. For example, the habit recommendation engine 312 may output a habit recommendation suggesting that the user improve their sleep habits and drink more water in order to improve the user's overall health. The habit recommendation engine 312 may be trained with the respective holistic scores received from the holistic analysis engine 306 to output respective habit recommendations.

The forecasting engine 314 may receive the skin condition value from the skin analysis engine 304 and the holistic score from the holistic analysis engine 306, and may output a skin forecast of the user based on the skin condition value of the user and the holistic score of the user. The skin forecast may generally be and/or include a prediction indicating how the user's skin may change over a certain period of time (e.g., days, weeks, months, years) based on the skin condition value and the holistic score. In certain aspects, the skin forecast may include a visual or graphical representation of the user's skin region featuring or otherwise representing the changes to the user's skin, as predicted by the forecasting engine 314. For example, the forecasting engine 314 may output a skin forecast indicating to a user that their skin will begin to wrinkle over the next year based on the user's skin condition value and the holistic score. Further in this example, the skin forecast may include a visual/graphical representation of the user's skin featuring the predicted appearance of the user's skin after one year, and may highlight or otherwise indicate the wrinkles mentioned in the textual portion of the skin forecast. The forecasting engine 314 may be trained with the respective skin condition values received from the skin analysis engine 304 and the respective holistic scores received from the holistic analysis engine 306 to output respective skin forecasts.

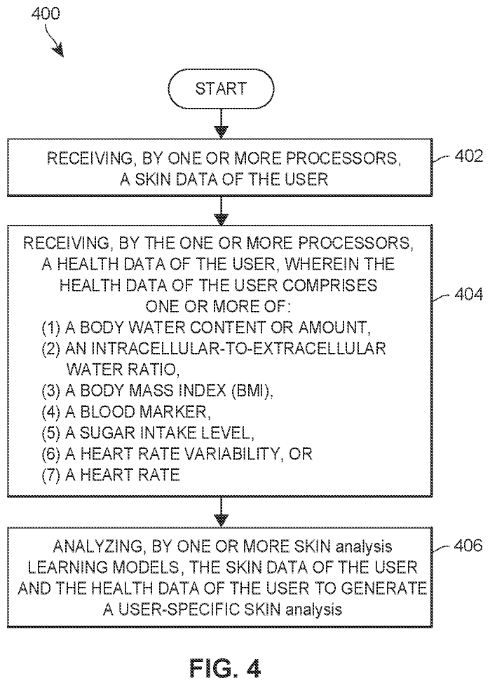

FIG. 4 illustrates an example user-specific skin analysis method 400 for generating a user-specific skin analysis, in accordance with various aspects disclosed herein. The user-specific data, as used with the method 400, and more generally as described herein, may be user responses/inputs received by a user computing device (e.g., user computing device 111c1) and/or images of a user's skin/skin region as captured by, for example, the user computing device. In some aspects, the user-specific data may comprise or refer to a plurality of responses/inputs/images such as a plurality of user responses collected by the user computing device while executing the analysis application (app), as described herein. Moreover, it is to be appreciated that the skin region of the user may be any suitable portion of the user's body, such as any or all of the user's arm, leg, torso, head, etc.

At block 402, the method 400 comprises receiving, by one or more processors (e.g., one or more processor(s) 104 of server(s) 102 and/or processors of a computer user device, such as a mobile device), a skin data of the user. More specifically, the skin data of the user may be received at an analysis application (app) executing on one or more processors. The skin data of the user may generally define a skin region of the user, and in certain aspects, may be skin image data of the user comprising image/video data of the skin region of the user. For example, the skin data may comprise image/video data that is a digital image as captured by an imaging device (e.g., an imaging device of user computing device 111c1 or 112c3). In this example, the image data may comprise pixel data of at least a portion of a skin region of the user.

However, in certain aspects, the skin data may include both image data and non-image data. Specifically, the non-image data may include user responses/inputs to a questionnaire presented as part of the execution of the analysis app. In some aspects, at least one of the one or more processors comprises at least one of a processor of a mobile device or a processor of a server.

At block 404, the method 400 comprises receiving, by the one or more processors, a health data of the user. The health data of the user may comprise one or more of: (1) a body water content or amount, (2) an intracellular-to-extracellular water ratio, (3) a body mass index (BMI), (4) a blood marker, (5) a sugar intake level, (6) a heart rate variability, or (7) a heart rate. In certain aspects, the health data of the user may be selected from the group consisting of: (1) the body water content or amount, (2) the intracellular-to-extracellular water ratio, (3) the body mass index (BMI), and/or mixtures thereof, in view of improved accuracy of skin analysis. In some aspects, the health data of the user may be selected from the group consisting of: (1) the body water content or amount, (2) the intracellular-to-extracellular water ratio, and/or mixtures thereof, in view of improved accuracy of skin analysis.

At block 406, the method 400 comprises analyzing, by one or more skin analysis learning models (e.g., skin analysis learning model 108), the skin data of the user and the health data of the user to generate a user-specific skin analysis. Particularly, the analysis app scalp may execute the one or more skin analysis learning models to output the user-specific skin analysis, which may correspond to one or more features of the skin region of the user. As previously described, in certain aspects, the one or more skin analysis models may comprise a plurality of skin analysis models, and each of the plurality of skin analysis models may be configured to receive input data and generate output data in a sequence. In certain aspects, the user-specific skin analysis comprises one or more of (1) a skin condition, (2) a holistic score to be defined based on at least skin and health data, (3) a skin product recommendation, (4) a skin product usage recommendation, (5) a supplemental product recommendation, (6) a supplemental product usage recommendation, (7) a habit recommendation, or (8) a skin forecast.

In various aspects, the user-specific skin analysis is rendered on a display screen of a computing device (e.g., user computing device 111c1). The rendering may include, as previously mentioned, instructions guiding the user to treat the condition identified based on the health data and skin data of the user. For example, the one or more skin analysis learning models may output a user-specific skin analysis indicating that the user has a sunburn. In this example, the one or more processors may additionally generate instructions for the user to purchase/apply aloe vera cream to soothe the sunburn (e.g., via the skin product recommendation engine 308), and to proactively apply sunscreen to the user's skin in order to avoid future burns (e.g., via the habit recommendation engine 312).

The user-specific skin analysis may be generated by a user computing device (e.g., user computing device 111c1) and/or by a server (e.g., server(s) 102). For example, in some aspects the server(s) 102, as described herein for FIG. 1, may analyze the user-specific data (skin data and health data of the user) remote from a user computing device to determine a user-specific skin analysis corresponding to the skin region of the user. For example, in such aspects the server or a cloud-based computing platform (e.g., server(s) 102) receives, across computer network 120, the user-specific data defining the skin region of the user comprising the skin data of the user and the health data of the user. The server or a cloud-based computing platform may then execute the AI based learning model (e.g., skin analysis learning model 108) and generate, based on output of the AI based learning model, the user-specific skin analysis. The server or a cloud-based computing platform may then transmit, via the computer network (e.g., computer network 120), the user-specific skin analysis to the user computing device for rendering on the display screen of the user computing device. For example, and in various aspects, the user-specific skin analysis may be rendered on the display screen of the user computing device in real-time or near-real time, during, or after receiving, the user-specific data defining the skin region of the user.

In various aspects, the user-specific treatment may comprise a textually-based treatment, a visual/image based treatment, and/or a virtual rendering of the user's skin region, e.g., displayed on the display screen of a user computing device (e.g., user computing device 111c1). Such a user-specific skin analysis may include a graphical representation of the user's skin region as annotated with one or more graphics or textual renderings corresponding to user-specific features (e.g., excessive skin irritation/redness, wrinkles, etc.).

Further, in certain aspects, the analysis app may receive an image of the user, and the image may depict the skin region of the user. In these aspects, the analysis app, executing on one or more processors, may generate a modified image based on the image that depicts how the skin region of the user is predicted to appear after following at least one of the recommendations included as part of the user-specific skin analysis. The analysis app may generate the modified image by manipulating one or more pixels of the image of the user based on the user-specific skin analysis. As an example, the analysis app may graphically render the user-specific skin analysis for display to a user, and the user-specific skin analysis may include a product/habit recommendation to increase the user's water intake and to apply an anti-aging cream to reduce wrinkles that the one or more skin analysis learning models determined are present in the user's skin region based on the user-specific data. In this example, the analysis app may generate a modified image of the user's skin region (as depicted in the image of the user) without wrinkles (or a reduced amount) by manipulating the pixel values of one or more pixels of the image (e.g., updating, smoothing, changing colors) of the user to alter the pixel values of pixels identified as containing pixel data representative of wrinkles present on the user's skin region to pixel values representative of non-wrinkled skin present in the user's skin region. As a result, the one or more processors executing the analysis app may render the modified image on the display screen of a computing device (e.g., user computing device 111c1).

In certain aspects, the skin data of the user is a first skin data of the user and the health data of the user is a first health data of the user. In these aspects, the one or more processors may receive the first skin data of the user and the first health data of the user at a first time, and a second skin data of the user and a second health data of the user at a second time. The one or more skin analysis models (e.g., skin analysis learning model 108) may analyze the second skin data of the user and the second health data of the user to generate, based on a comparison of the second skin data of the user and the second health data of the user to the first skin data of the user and the first health data of the user, a new user-specific skin analysis. In this manner, the analysis app, as executed on the one or more processors, may track the changes to the skin region (and the user's skin generally) of the user over time. The new user-specific analysis may be identical or different from the user-specific skin analysis based on the first health data and the first skin data. For example, the user-specific skin analysis may include a recommendation suggesting that a user increase the amount of sleep they receive on a nightly basis. In this example, the new user-specific skin analysis may not include that suggestion because, between the first time and the second time, the user may have gotten the recommended amount of sleep, and as a result, the user's skin region may not include features indicative of a lack of sleep.

As referred to herein in various aspects, an AI based learning model (e.g., skin analysis learning model 108) may be trained with skin data and health data of respective individuals to output the user-specific skin analysis. More specifically, in certain aspects, the one or more skin analysis learning models may each be trained with digital image data of a plurality of training images depicting skin regions of skin of respective individuals and health data of the respective individuals, such that the one or more skin analysis models are configured to output one or more skin analysis corresponding to one or more features of skin regions of the respective individuals. Moreover, in various aspects, the one or more skin analysis learning models may each be trained with a plurality of training images and a plurality of non-image training data corresponding to the respective individuals.

For example, a first set of training data corresponding to a first respective individual may include skin data representing damaged (e.g., sunburned) skin and health data indicating that the first respective individual is relatively healthy (e.g., low BMI, healthy sugar levels, healthy heart rate, etc.). Further in this example, a second set of training data corresponding to a second respective individual may include skin data representing older (e.g., wrinkled) skin and health data indicating that the second respective individual is moderately unhealthy (e.g., moderate/high BMI, moderate sugar levels, slightly elevated heart rate, etc.). Finally, in this example, a third set of data corresponding to a third respective individual may include skin data representing healthy skin and health data indicating that the third respective individual is very unhealthy (e.g., high BMI, high sugar levels, very elevated heart rate, etc.). In this example, the one or more skin analysis learning models may be trained with the first, second, and third sets of training data to better identify/correlate each of the conditions represented in the respective sets of training data.

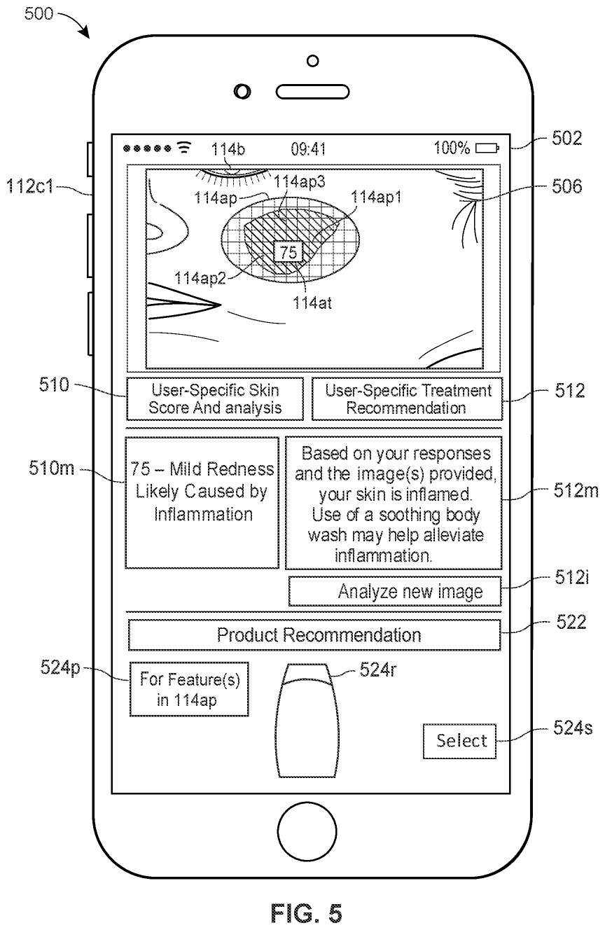

FIG. 5 illustrates an example user interface 500 as rendered on a display screen 502 of a user computing device (e.g., user computing device 112c1) in accordance with various aspects disclosed herein. For example, as shown in the example of FIG. 5, the user interface 500 may be implemented or rendered via an application (app) executing on user computing device 112c1. For example, as shown in the example of FIG. 5, user interface 500 may be implemented or rendered via a native app executing on user computing device 112c1. In the example of FIG. 5, user computing device 112c1 is a user computing device as described for FIG. 1, e.g., where 112c1 is illustrated as an APPLE iPhone that implements the APPLE iOS operating system and that has display screen 502. User computing device 112c1 may execute one or more native applications (apps) on its operating system, including, for example, the analysis app, as described herein. Such native apps may be implemented or coded (e.g., as computing instructions) in a computing language (e.g., SWIFT) executable by the user computing device operating system (e.g., APPLE iOS) by the processor of user computing device 112c1.

Additionally, or alternatively, user interface 500 may be implemented or rendered via a web interface, such as via a web browser application, e.g., Safari and/or Google Chrome app(s), or other such web browser or the like.

As shown in the example of FIG. 5, the user interface 500 comprises a graphical representation (e.g., of image 114b) of a user's skin region 506. The image 114b may comprise an image of the user (or graphical representation 506 thereof) comprising pixel data (e.g., pixel data 114ap) of at least a portion of the skin region of the user, as described herein. In the example of FIG. 5, the graphical representation (e.g., of image 114b) of the user's skin region 506 is annotated with one or more graphics (e.g., area of pixel data 114ap) or textual rendering(s) (e.g., text 114at) corresponding to various features identifiable within the pixel data comprising a portion of the skin region of the user. For example, the area of pixel data 114ap may be annotated or overlaid on top of the image of the user (e.g., image 114b) to highlight the area or feature(s) identified within the pixel data (e.g., feature data and/or raw pixel data) by the AI based learning model (e.g., skin analysis learning model 108). In the example of FIG. 5, the area of pixel data 114ap indicates features, as defined in pixel data 114ap, including skin redness/irritation indicative of inflammation (e.g., for pixels 114ap1-3), and may indicate other features shown in area of pixel data 114ap (e.g., skin wrinkles, sunburn, etc.), as described herein. In various aspects, the pixels identified as the specific features (e.g., pixels 114ap1-3), may be highlighted or otherwise annotated when rendered on display screen 502.

As an example, the first pixel 114ap1 may represent an area of the user's skin that features an elevated level of dryness and/or irritation relative to the user's healthy skin included within the image 114b and/or relative to healthy skin images of respective individuals used to train the AI based learning model (e.g., skin analysis learning model 108). The second pixel 114ap2 may represent an area of the user's skin that features an elevated level of inflammation relative to the user's healthy skin included within the image 114b and/or relative to healthy skin images of respective individuals used to train the AI based learning model (e.g., skin analysis learning model 108). The third pixel 114ap3 may represent an area of the user's skin that features an elevated level of redness relative to the user's healthy skin included within the image 114b and/or relative to healthy skin images of respective individuals used to train the AI based learning model (e.g., skin analysis learning model 108). Thus, in this example, when the one or more skin analysis learning models evaluate each of these pixels (in addition to the other pixels in the area of pixel data 114ap and included within the rest of the image 114b) the models may output a user-specific skin analysis (e.g., user-specific skin score and analysis 510) that informs the user that their skin features these characteristics (e.g., dryness/irritation, redness, etc.) likely as a result of inflammation.

The textual rendering (e.g., text 114at) shows a user-specific skin score (e.g., 75 for pixels 114ap1-3) which may indicate that the user has an above-average skin score (of 75) as a result of the inflammation. The 75 score indicates that the user has a relatively low degree of redness as a result of inflammation present on the user's skin region, such that the user would likely benefit from washing their skin with a soothing body wash specifically designed to improve their skin health/quality/condition (e.g., reduce the amount/degree of redness/inflammation). It is to be understood that other textual rendering types or values are contemplated herein, where textual rendering types or values may be rendered, for example, such as a skin quality score, a holistic score, or the like. Additionally, or alternatively, color values may be used and/or overlaid on a graphical representation (e.g., graphical representation of the user's skin region 506) shown on user interface 500 to indicate a degree or quality of a given score, e.g., a low score of 25 or a high score of 90. The scores may be provided as raw scores, absolute scores, percentage based scores, and/or any other suitable presentation style. Additionally, or alternatively, such scores may be presented with textual or graphical indicators indicating whether or not a score is representative of positive results (good skin health), negative results (poor skin health), or acceptable results (average or acceptable skin health/skin care).

User interface 500 may also include or render a user-specific skin score and analysis 510. In the aspect of FIG. 5, the user-specific skin score and analysis 510 comprises a message 510m to the user designed to indicate the user-specific skin analysis and/or skin/holistic score to the user, along with a brief description of any reasons resulting in the user-specific skin analysis and/or skin/holistic score. As shown in the example of FIG. 5, the message 510m indicates to a user that the user-specific skin score is “75” and further indicates to the user that the user-specific skin analysis corresponds to the user-specific skin score because the user's skin region has “mild redness likely caused by inflammation.”

User interface 500 may also include or render a user-specific treatment recommendation 512. In the aspect of FIG. 5, the user-specific treatment recommendation 512 may be and/or include any of a skin product recommendation (via the skin product recommendation engine 308), a supplementary product recommendation (via the supplementary product recommendation engine 310), a habit recommendation (via the habit recommendation engine 312), a skin forecast (via the forecasting engine 314), and/or any other suitable recommendation/value/score as described herein or combinations thereof. The user-specific treatment recommendation 512 may comprise a message 512m to the user designed to address at least one feature identifiable within the user-specific data defining the skin region of the user.

As shown in the example of FIG. 5, message 512m recommends to the user to wash their skin with a soothing body wash to improve their skin health/quality/condition by reducing the redness resulting from inflammation. The soothing body wash recommendation can be made based on the above-average user-specific skin score (e.g., 75) suggesting that the image of the user depicts a mild amount of redness resulting from inflammation, where the soothing body wash product is designed to address inflammation/redness detected or classified in the pixel data of image 114b or otherwise predicted based on the user-specific data (e.g., the skin data and the health data) of the user. The product recommendation can be correlated to the identified feature within the user-specific data and/or the pixel data, and the user computing device 112c1 and/or server(s) 102 can be instructed to output the product recommendation (via the skin/supplementary product recommendation engines 308/310) when the feature (e.g., mild skin redness/inflammation) is identified.

The user interface 500 may also include or render a section for a product recommendation 522 for a manufactured product 524r (e.g., soothing body wash, as described above). The product recommendation 522 may correspond to the user-specific treatment recommendation 512, as described above. For example, in the example of FIG. 5, the user-specific treatment recommendation 512 may be displayed on the display screen 502 of user computing device 112c1 with instructions (e.g., message 512m) for treating, with the manufactured product (manufactured product 524r (e.g., soothing body wash)) at least one feature (e.g., above-average user-specific skin score of 75 related to redness/inflammation at pixels 114ap1-3) predicted and/or identifiable based on the user-specific data (including the pixel data 114ap) comprising pixel data of at least a portion of a skin region of the user. The features predicted or identified are indicated and annotated (524p) on the user interface 500.

As shown in FIG. 5, the user interface 500 recommends a product (e.g., manufactured product 524r (e.g., soothing body wash)) based on the user-specific treatment recommendation 512. In the example of FIG. 5, the output or analysis of the user-specific data by the AI based learning model (e.g., skin analysis learning model 108), e.g., user-specific skin score and analysis 510 and/or its related values (e.g., 75 user-specific skin score) or related pixel data (e.g., 114ap1, 114ap2, and/or 114ap3), and/or the user-specific treatment recommendation 512, may be used to generate or identify recommendations for corresponding product(s). Such recommendations may include products such as body wash, anti-aging products, anti-oxidant products, anti-wrinkle products, hydration products, anti-inflammatory products, shampoo, conditioner, and the like to address the user-specific issue as detected or predicted from the user-specific data.

User interface 500 may further include a selectable UI button 524s to allow the user (e.g., the user of image 114b) to select for purchase or shipment the corresponding product (e.g., manufactured product 524r). In some aspects, selection of selectable UI button 524s may cause the recommended product(s) to be shipped to the user and/or may notify a third party that the individual is interested in the product(s). For example, either user computing device 112c1 and/or the server(s) 102 may initiate, based on the user-specific skin score and analysis 510 and/or the user-specific treatment recommendation 512, the manufactured product 524r (e.g., soothing body wash) for shipment to the user. In such aspects, the product may be packaged and shipped to the user.

In various aspects, a graphical representation (e.g., graphical representation of the user's skin region 506), with graphical annotations (e.g., area of pixel data 114ap), textual annotations (e.g., text 114at), and the user-specific skin score and analysis 510 and the user-specific treatment recommendation 512 may be transmitted, via the computer network (e.g., from a server 102 and/or one or more processors) to the user computing device 112c1, for rendering on the display screen 502. In other aspects, no transmission to the server of the user's specific image occurs, where the user-specific skin score and analysis 510 and the user-specific treatment recommendation 512 (and/or product specific recommendation) may instead be generated locally, by the AI based learning model (e.g., skin analysis learning model 108) executing and/or implemented on the user's mobile device (e.g., user computing device 112c1) and rendered, by a processor of the mobile device, on display screen 502 of the mobile device (e.g., user computing device 112c1).

In some aspects, any one or more of graphical representations (e.g., graphical representation of the user's skin region 506), with graphical annotations (e.g., area of pixel data 114ap), textual annotations (e.g., text 114at), user-specific skin score and analysis 510, user-specific treatment recommendation 512, and/or product recommendation 522 may be rendered (e.g., rendered locally on display screen 502) in real-time or near-real time during or after receiving, the user-specific data. In aspects where the user-specific data is analyzed by a server(s) 102, the user-specific data may be transmitted and analyzed in real-time or near real-time by the server(s) 102.

In some aspects, the user may provide new user-specific data that may be transmitted to the server(s) 102 for updating, retraining, or reanalyzing by the skin analysis learning model 108. In other aspects, new user-specific data may be locally received on computing device 112c1 and analyzed, by the skin analysis learning model 108, on the computing device 112c1.

In addition, as shown in the example of FIG. 5, the user may select selectable button 512i for reanalyzing (e.g., either locally at computing device 112c1 or remotely at the server(s) 102) new user-specific data. Selectable button 512i may cause the user interface 500 to prompt the user to input/attach for analyzing new user-specific data. The server(s) 102 and/or a user computing device such as user computing device 112c1 may receive the new user-specific data comprising data that defines a skin region of the user. Specifically, the new user-specific data may be received/captured by the user computing device 112c1 (e.g., via an integrated digital camera of the user computing device 112c1). A new image (e.g., similar to image 114b) included as a portion of the new user-specific data may comprise pixel data of a portion of a skin region of the user. The AI based learning model (e.g., skin analysis learning model 108), executing on the memory of the computing device (e.g., server(s) 102), may analyze the new user-specific data received/captured by the user computing device 112c1 to generate a new user-specific skin analysis. The computing device (e.g., server(s) 102) may generate, a new user-specific skin analysis based on a comparison of the new user-specific data and the user-specific data. For example, the new user-specific skin analysis may include a new graphical representation including graphics and/or text (e.g., showing a new user-specific skin score, e.g., 85, after the user washed their skin using a soothing body wash). The new user-specific skin analysis may include additional quality scores, e.g., that the user has successfully washed their skin to reduce skin redness/inflammation, as detected with the new user-specific data. A comment may include that the user needs to correct additional features detected within the new user-specific data, e.g., skin dryness, by applying an additional product, e.g., moisturizing lotion.

In various aspects, the new user-specific skin analysis and/or a new user-specific treatment recommendation may be transmitted via the computer network, from server(s) 102, to the user computing device of the user for rendering on the display screen 502 of the user computing device (e.g., user computing device 112c1).

In other aspects, no transmission to the server of the user's new user-specific data occurs, where the new user-specific skin analysis and/or the new user-specific treatment recommendation (and/or product/habit specific recommendation) may instead be generated locally, by the AI based learning model (e.g., skin analysis learning model 108) executing and/or implemented on the user's mobile device (e.g., user computing device 112c1) and rendered, by a processor of the mobile device, on a display screen 502 of the mobile device 112c1.

### EXAMPLES

A user-specific skin analysis method for generating a user-specific skin analysis, was conducted by using the user-specific skin analysis system shown in FIG. 1, especially by using health data being a body water content or amount of the user, together with a skin image data of the user. User-specific skin analysis being skin condition and skin forecast, especially pigmented spots and forecasting of pigmented spots were provided on a display screen of a computing device. The method and the system of the present invention provide improved accuracy of skin analysis, compared to those using only skin data without health data, and also compared to those using other health data such as percent body fat.

### Aspects of the Disclosure

The following aspects are provided as examples in accordance with the disclosure herein and are not intended to limit the scope of the disclosure.

1. A user-specific skin analysis method for generating a user-specific skin analysis, the method comprising: receiving, by one or more processors, a skin data of the user; receiving, by the one or more processors, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and analyzing, by one or more skin analysis learning models, the skin data of the user and the health data of the user to generate, a user-specific skin analysis.

analysis2. The method of aspects 1, wherein the health data of the user is selected from the group consisting of: the body water content or amount; the intracellular-to-extracellular water ratio; the body mass index (BMI); and mixtures thereof.

3. The method of any of aspects 1-2, wherein the health data of the user is selected from the group consisting of: the body water content or amount; the intracellular-to-extracellular water ratio; and mixtures thereof.

4. The method of any of aspects 1-3, wherein the one or more skin analysis learning models are trained with skin data and health data of respective individuals to output the user-specific skin analysis.

5. The method of any of aspects 1-4, further comprising: rendering, by the one or more processors, the user-specific skin analysis on a display screen of a computing device.

6. The method of any of aspects 1-5, further comprising: receiving, by the one or more processors, an image depicting a skin region of the user; generating, by the one or more processors, a modified image based on the image, the modified image depicting how the skin region of the user is predicted to appear after following at least one of the recommendations; and rendering, by the one or more processors, the modified image on the display screen of the computing device.

7. The method of any of aspects 1-6, wherein the skin data of the user is a first skin data of the user and the health data of the user is a first health data of the user, the method further comprising: receiving, by the one or more processors, the first skin data of the user and the first health data of the user at a first time; receiving, by the one or more processors, a second skin data of the user and a second health data of the user at a second time; analyzing, by the one or more skin analysis models, the second skin data of the user and the second health data of the user; and generating, based on a comparison of the second skin data of the user and the second health data of the user to the first skin data of the user and the first health data of the user, a new user-specific skin analysis.

8. The method of any of aspects 1-7, wherein at least one of the one or more processors comprises at least one of a processor of a mobile device or a processor of a server.

9. The method of any of aspects 1-8, wherein the skin data of the user is skin image data of the user.

10. A user-specific skin analysis system configured to generate a user-specific skin analysis, the user-specific skin analysis system comprising: one or more processors; and an analysis application (app) comprising computing instructions configured to execute on the one or more processors; and one or more skin analysis learning models, accessible by the analysis app, wherein the computing instructions of the analysis app when executed by the one or more processors, cause the one or more processors to: receive a skin data of the user, receive a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate, and analyze, by the one or more skin analysis learning models, the skin data of the user and the health data of the user to generate, a user-specific skin analysis.

analysis 11. The system of aspect 10, wherein the health data of the user is selected from the group consisting of: the body water content or amount; the intracellular-to-extracellular water ratio; the body mass index (BMI); and mixtures thereof.

12. The system of any of aspects 10-11, wherein the health data of the user is selected from the group consisting of: the body water content or amount; the intracellular-to-extracellular water ratio; and mixtures thereof.

13. The system of any of aspects 10-12, wherein the one or more skin analysis learning models are trained with skin data and health data of respective individuals to output the user-specific skin analysis.

14. The system of any of aspects 10-14, wherein the computing instructions, when executed by the one or more processors, further cause the one or more processors to: render the user-specific skin analysis on a display screen of a computing device.

15. The system of any of aspects 10-14, wherein the computing instructions, when executed by the one or more processors, further cause the one or more processors to: receive an image depicting a skin region of the user; generate a modified image based on the image, the modified image depicting how the skin region of the user is predicted to appear after following at least one of the recommendations; and render the modified image on the display screen of the computing device.

16. The system of any of aspects 10-15, wherein the skin data of the user is a first skin data of the user and the health data of the user is a first health data of the user, the computing instructions, when executed by the one or more processors, further cause the one or more processors to: receive the first skin data of the user and the first health data of the user at a first time; receive a second skin data of the user and a second health data of the user at a second time; analyze, by the one or more skin analysis models, the second skin data of the user and the second health data of the user; and generate, based on a comparison of the second skin data of the user and the second health data of the user to the first skin data of the user and the first health data of the user, a new user-specific skin analysis.

17. The system of any of aspects 10-16, wherein at least one of the one or more processors comprises at least one of a processor of a mobile device or a processor of a server.

18. The system of any of aspects 10-17, wherein the skin data of the user is skin image data of the user.

19. A tangible, non-transitory computer-readable medium storing instructions for generating a user-specific skin analysis, that when executed by one or more processors cause the one or more processors to: receive, at an analysis application (app) executing on one or more processors, a skin data of the user; receive, at the analysis app, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and analyze, by the one or more skin analysis learning models accessible by the analysis app, the skin data of the user and the health data of the user to generate, a user-specific skin analysis.

analysis 20. The tangible, non-transitory computer-readable medium of aspects 19, wherein the skin data of the user is skin image data of the user.

### Additional Considerations

Although the disclosure herein sets forth a detailed description of numerous different aspects, it should be understood that the legal scope of the description is defined by the words of the claims set forth at the end of this patent and equivalents. The detailed description is to be construed as exemplary only and does not describe every possible aspect since describing every possible aspect would be impractical. Numerous alternative aspects may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example aspects, the processor or processors may be located in a single location, while in other aspects the processors may be distributed across a number of locations.

The performance of certain of the operations may be distributed among the one or more processors, not only residing within a single machine, but deployed across a number of machines. In some example aspects, the one or more processors or processor-implemented modules may be located in a single geographic location (e.g., within a home environment, an office environment, or a server farm). In other aspects, the one or more processors or processor-implemented modules may be distributed across a number of geographic locations.

This detailed description is to be construed as exemplary only and does not describe every possible aspect, as describing every possible aspect would be impractical, if not impossible. A person of ordinary skill in the art may implement numerous alternate aspects, using either current technology or technology developed after the filing date of this application.

Those of ordinary skill in the art will recognize that a wide variety of modifications, alterations, and combinations can be made with respect to the above described aspects without departing from the scope of the invention, and that such modifications, alterations, and combinations are to be viewed as being within the ambit of the inventive concept.

The patent claims at the end of this patent application are not intended to be construed under 35 U.S.C. § 112(1) unless traditional means-plus-function language is expressly recited, such as “means for” or “step for” language being explicitly recited in the claim(s). The systems and methods described herein are directed to an improvement to computer functionality, and improve the functioning of conventional computers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A user-specific skin analysis method for generating a user-specific skin analysis, the method comprising:
receiving, by one or more processors, a skin data of the user;
receiving, by the one or more processors, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and
analyzing, by one or more skin analysis learning models, the skin data of the user and the health data of the user to generate a user-specific skin analysis.

2. The method of claim 1, wherein the health data of the user is selected from the group consisting of: the body water content or amount; the intracellular-to-extracellular water ratio; the body mass index (BMI); and mixtures thereof.

3. The method of claim 1, wherein the health data of the user is selected from the group consisting of: the body water content or amount; the intracellular-to-extracellular water ratio; and mixtures thereof.

4. The method of claim 1, wherein the one or more skin analysis learning models are trained with skin data and health data of respective individuals to output the user-specific skin analysis.

5. The method of claim 1, further comprising:
rendering, by the one or more processors, the user-specific skin analysis on a display screen of a computing device.

6. The method of claim 1, further comprising:
receiving, by the one or more processors, an image depicting a skin region of the user;
generating, by the one or more processors, a modified image based on the image, the modified image depicting how the skin region of the user is predicted to appear after following at least one of the recommendations; and
rendering, by the one or more processors, the modified image on the display screen of the computing device.

7. The method of claim 1, wherein the skin data of the user is a first skin data of the user and the health data of the user is a first health data of the user, the method further comprising:
receiving, by the one or more processors, the first skin data of the user and the first health data of the user at a first time;
receiving, by the one or more processors, a second skin data of the user and a second health data of the user at a second time;
analyzing, by the one or more skin analysis models, the second skin data of the user and the second health data of the user; and
generating, based on a comparison of the second skin data of the user and the second health data of the user to the first skin data of the user and the first health data of the user, a new user-specific skin analysis.

8. The method of claim 1, wherein at least one of the one or more processors comprises at least one of a processor of a mobile device or a processor of a server.

9. The method of claim 1, wherein the skin data of the user is skin image data of the user.

10. A user-specific skin analysis system configured to generate a user-specific skin analysis, the user-specific skin analysis system comprising:
one or more processors; and
an analysis application (app) comprising computing instructions configured to execute on the one or more processors; and
one or more skin analysis learning models, accessible by the analysis app,
wherein the computing instructions of the analysis app when executed by the one or more processors, cause the one or more processors to:
receive a skin data of the user,
receive a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate, and
analyze, by the one or more skin analysis learning models, the skin data of the user and the health data of the user to generate a user-specific skin analysis.

11. The system of claim 10, wherein the health data of the user is selected from the group consisting of: the body water content or amount; the intracellular-to-extracellular water ratio;
the body mass index (BMI); and mixtures thereof.

12. The system of claim 10, wherein the health data of the user is selected from the group consisting of: the body water content or amount; the intracellular-to-extracellular water ratio;
and mixtures thereof.

13. The system of claim 10, wherein the one or more skin analysis learning models are trained with skin data and health data of respective individuals to output the user-specific skin analysis.

14. The system of claim 10, wherein the computing instructions, when executed by the one or more processors, further cause the one or more processors to:
render the user-specific skin analysis on a display screen of a computing device.

15. The system of claim 10, wherein the computing instructions, when executed by the one or more processors, further cause the one or more processors to:
receive an image depicting a skin region of the user;
generate a modified image based on the image, the modified image depicting how the skin region of the user is predicted to appear after following at least one of the recommendations; and
render the modified image on the display screen of the computing device.

16. The system of claim 10, wherein the skin data of the user is a first skin data of the user and the health data of the user is a first health data of the user, the computing instructions, when executed by the one or more processors, further cause the one or more processors to:
receive the first skin data of the user and the first health data of the user at a first time;
receive a second skin data of the user and a second health data of the user at a second time;
analyze, by the one or more skin analysis models, the second skin data of the user and the second health data of the user; and
generate, based on a comparison of the second skin data of the user and the second health data of the user to the first skin data of the user and the first health data of the user, a new user-specific skin analysis.

17. The system of claim 10, wherein at least one of the one or more processors comprises at least one of a processor of a mobile device or a processor of a server.

18. The system of claim 10, wherein the skin data of the user is skin image data of the user.

19. A tangible, non-transitory computer-readable medium storing instructions for generating a user-specific skin analysis, that when executed by one or more processors cause the one or more processors to:
receive, at an analysis application (app) executing on one or more processors, a skin data of the user;
receive, at the analysis app, a health data of the user, wherein the health data of the user comprises one or more of: (1) a body water content or amount, (2) an intracellular-to-extracellular water ratio, (3) a body mass index (BMI), (4) a blood marker, (5) a sugar intake level, (6) a heart rate variability, or (7) a heart rate; and
analyze, by the one or more skin analysis learning models accessible by the analysis app, the skin data of the user and the health data of the user to generate a user-specific skin analysis.

20. The tangible, non-transitory computer-readable medium of claim 19, wherein the skin data of the user is skin image data of the user.
